(19) United States
(12) Patent Application Publication
(10) Pub. No.: US 2026/0026741 A1
(43) Pub. Date: Jan. 29, 2026

(54) SYSTEM FOR PROVIDING SLEEP STATE ANALYSIS INFORMATION AND IMPROVEMENT GUIDE, AND METHOD THEREFOR

(71) Applicant: BRLAB INC., Seoul (KR)

(72) Inventors: Kwang Suk PARK, Seoul (KR); Byung Hun CHOI, Seoul (KR); Hyun Bin KWON, Seoul (KR); Jong Hyeok JEONG, Seoul (KR)

(21) Appl. No.: 19/350,546

(22) Filed: Oct. 6, 2025

Related U.S. Application Data
(63) Continuation of application No. PCT/KR2024/004847, filed on Apr. 11, 2024.

(30) Foreign Application Priority Data
Apr. 10, 2023 (KR) 10-2023-0047109
Apr. 11, 2024 (KR) 10-2024-0048621

(51) Int. Cl. A61B 5/00

(52) U.S. Cl. CPC A61B 5/4809; A61B 5/4812; A61B 5/4815; A61B 5/6892; A61B 5/7271

(57) ABSTRACT

The present invention relates to a system for providing sleep state analysis information and an improvement guide, and method therefor. More particularly, a system for providing sleep information analysis information and an improvement guide, according to an embodiment of the present invention, comprises: at least one layer unit that has a plurality of components; a detection unit that is provided in the layer unit, and detects biometric information of a user; a memory unit that stores the biometric information of the user detected by the detection unit; and an analysis unit that analyzes a sleep state of the user on the basis of the biometric information of the user stored in the memory unit, and generates sleep state analysis information of the user.

### CROSS-REFERENCE TO RELATED APPLICATIONS

This application is a Continuation of Application No. PCT/KR2024/004847, filed on Apr. 11, 2024, which in turn claims the benefit of Korean Patent Applications No. 10-2023-0047109,filed on Apr. 10, 2023, and No. 10-2024-0048621, filed on Apr. 11, 2024. The entire disclosures of all these applications are hereby incorporated by reference.

### TECHNICAL FIELD

The present invention relates to a system for providing sleep state analysis information and an improvement guide, and a method therefor. More specifically, the present invention relates to a system for providing sleep state analysis information and an improvement guide, and a method therefor, which can analyze the sleep state of a user on the basis of biometric information detected while the user sleeps, and provide sleep state analysis information, and also provide an improvement guide that can change the sleep state.

### BACKGROUND ART

Recently, as the level and quality of living of people are improved, interest in ‘good sleep’ is increased, and in particular, the industry that provides various devices or services inducing sleep using latest scientific techniques is growing remarkably. Accordingly, many devices and services improving sleep quality, such as sleep care services that guide environments, habits, postures, and the like of sleep through consulting with experts, services that monitor sleep states by sensing breathing sounds or the like of a user when the user wears a wearable device, and the like, are commercialized. Specifically, various devices and services that receive information such as body information, state information, sleep habits, and like of a user, or collect information such as the state information or sleep habits of a user through individual equipment connected to the user in a contact or non-contact manner, and perform a function of inducing ‘good sleep’ on the basis of the information of the user are developed, and through this, sleep states can be induced based on the state information of the user.

Equipment measuring the state information, biometric information, and the like of a user is essential to accurately determine a sleep state of the user, and the user should wear separate equipment for this this purpose, and wearing such separate equipment inevitably invites inconvenience when the user sleeps. In addition, conventionally, when a result of monitoring the sleep state of a user through such equipment is provided to the user, the result is provided as information such as classification of sleep state and non-sleep state, duration of the classified sleep state and non-sleep state, and the like. However, there is a disadvantage in that it is difficult for the user to grasp details of the sleep states of the user and it is difficult to devise specific measures for improving his or her sleep state with information of such types.

The present invention has been proposed to overcome the limitations of the prior art, and provides a system for providing sleep state analysis information and an improvement guide, and a method therefor, which can analyze the sleep state of a user more accurately on the basis of detected biometric information within a range that does not disturb the sleep of the user while the user sleeps, and provide an improvement guide that can change the sleep state of the user.

### DISCLOSURE OF INVENTION

### Technical Problem

An object of the present invention is to provide a system for providing sleep state analysis information and an improvement guide, and a method therefor, which can analyze the sleep state of a user more accurately on the basis of detected biometric information within a range that does not disturb the sleep of the user while the user sleeps, and provide analysis information.

In addition, another object of the present invention is to provide a system for providing sleep state analysis information and an improvement guide, and a method therefor, which can analyze the sleep state of a user in a short term or long term and provide analysis information.

In addition, another object of the present invention is to provide a system for providing sleep state analysis information and an improvement guide, and a method therefor, which can provide an improvement guide that can change the sleep state of a user with reference to detected biometric information of a user.

Meanwhile, technical problems of the present invention are not limited to the technical problems mentioned above, and unmentioned other technical problems can be clearly understood by those skilled in the art from the following descriptions.

### Technical Solution

To accomplish the above objects, according to one aspect of the present invention, there is provided a system for providing sleep state analysis information and an improvement guide, the system comprising: at least one layer unit having a plurality of components, a detection unit provided in the layer unit to detect biometric information of a user, a memory unit for storing the biometric information of the user detected by the detection unit, and an analysis unit for analyzing a sleep state of the user on the basis of the biometric information of the user stored in the memory unit, and generating sleep state analysis information of the user.

In addition, in the system for providing sleep state analysis information and an improvement guide according to the present invention, the detection unit includes at least one sensor among a pressure sensor, a vibration sensor, a position sensor, an acoustic sensor, an infrared sensor, a motion sensor, a face recognition sensor, a biometric recognition sensor, and a fingerprint recognition sensor.

In addition, in the system for providing sleep state analysis information and an improvement guide according to the present invention, the biometric information of the user collected by the detection unit is stored in the memory unit to be mapped to each of a plurality of users.

In addition, in the system for providing sleep state analysis information and an improvement guide according to the present invention, the analysis unit analyzes the biometric information of the user stored in the memory unit in a short term, calculates a result thereof as a numerical index, and generates the sleep state analysis information of the user by including the numerical index.

In addition, in the system for providing sleep state analysis information and an improvement guide according to the present invention, the analysis unit analyzes the biometric information of the user stored in the memory unit in a long term, determines a sleep state type of the user on the basis of a difference in the sleep state during an analysis period, and generates the sleep state analysis information of the user by including the sleep state type.

In addition, in the system for providing sleep state analysis information and an improvement guide according to the present invention, the sleep state analysis information of the user analyzed by the analysis unit is stored in the memory unit.

In addition, in the system for providing sleep state analysis information and an improvement guide according to the present invention, the sleep state analysis information of the user analyzed by the analysis unit is stored in the memory unit to be mapped to each of a plurality of users.

In addition, in the system for providing sleep state analysis information and an improvement guide according to the present invention, the analysis unit generates an improvement guide that can change the sleep state of the user on the basis of the biometric information of the user and the sleep state analysis information of the user stored in the memory unit.

In addition, the system for providing sleep state analysis information and an improvement guide according to the present invention further comprises a feedback receiving unit for monitoring information on whether a user executes the improvement guide generated by the analysis unit.

In addition, in the system for providing sleep state analysis information and an improvement guide according to the present invention, information on whether the user executes the improvement guide, which is monitored through the feedback receiving unit, is stored in the memory unit.

According to another aspect of the present invention, there is provided a method of providing sleep state analysis information and an improvement guide, which is performed in a system including a layer unit, a detection unit, a memory unit, and an analysis unit to provide the sleep state analysis information and the improvement guide, the method comprising the steps of: detecting biometric information of a user from the detection unit provided in the layer unit; storing the biometric information of the user detected by the detection unit in the memory unit; and analyzing a sleep state of the user on the basis of the biometric information of the user stored in the memory unit, and generating sleep state analysis information of the user, by the analysis unit.

In addition, in the method of providing sleep state analysis information and an improvement guide according to the present invention, the step of detecting biometric information of a user from the detection unit is performed by the detection unit including at least one sensor among a pressure sensor, a vibration sensor, a position sensor, an acoustic sensor, an infrared sensor, a motion sensor, a face recognition sensor, a biometric recognition sensor, and a fingerprint recognition sensor.

In addition, in the method of providing sleep state analysis information and an improvement guide according to the present invention, the step of storing the biometric information of the user in the memory unit is performed to store the biometric information of the user collected by the detection unit to be mapped to each of a plurality of users.

In addition, in the method of providing sleep state analysis information and an improvement guide according to the present invention, the step of generating sleep state analysis information of the user by the analysis unit includes the steps of: analyzing the biometric information of the user stored in the memory unit in a short term; calculating a result of analyzing in a short term as a numerical index; and generating the sleep state analysis information of the user by including the calculated numerical index.

In addition, in the method of providing sleep state analysis information and an improvement guide according to the present invention, the step of generating sleep state analysis information of the user by the analysis unit includes the steps of: analyzing the biometric information of the user stored in the memory unit in a long term; determining a sleep state type of the user on the basis of the difference in the sleep state during the long-term analysis period; and generating the sleep state analysis information of the user by including the determined sleep state type of the user.

In addition, the method of providing sleep state analysis information and an improvement guide according to the present invention further comprises, after the step of generating sleep state analysis information of the user by the analysis unit, the step of storing the analyzed sleep state analysis information of the user in the memory unit.

In addition, in the method of providing sleep state analysis information and an improvement guide according to the present invention, the step of storing the sleep state analysis information of the user in the memory unit is performed to store the sleep state analysis information of the user to be mapped to each of a plurality of users.

In addition, the method of providing sleep state analysis information and an improvement guide according to the present invention further comprises, after the step of storing the sleep state analysis information of the user in the memory unit, the step of generating an improvement guide that can change the sleep state of the user on the basis of the biometric information of the user and the sleep state analysis information of the user stored in the memory unit, by the analysis unit.

In addition, the method of providing sleep state analysis information and an improvement guide according to the present invention further comprises, after the step of generating an improvement guide by the analysis unit, the step of monitoring information on whether the user executes the improvement guide generated by the analysis unit, by a feedback receiving unit.

In addition, the method of providing sleep state analysis information and an improvement guide according to the present invention further comprises, after the step of monitoring information on whether the user executes the improvement guide by the feedback receiving unit, the step of storing information on whether the user executes the improvement guide monitored by the feedback receiving unit in the memory unit

### ADVANTAGEOUS EFFECTS

According to the present invention, as the sleep state of a user is analyzed on the basis of biometric information of the user detected through a detection unit in a mattress while the user sleeps, it is possible to analyze the sleep state of the user more accurately and provide analysis information within a range that does not disturb the sleep of the user.

In addition, according to the present invention, as the sleep state of a user is analyzed in a short term on the basis of detected biometric information of the user and provided as a numerical index, a degree of affecting the activities of the user after the sleep can be intuitively recognized.

In addition, according to the present invention, as the sleep state of a user is analyzed in a long term on the basis of detected biometric information of the user, and differences between the sleep states within a period are provided, information on the sleep type of the user may also be provided.

Furthermore, according to the present invention, an improvement guide that can change the sleep state of a user can be provided with reference to the detected biometric information of the user and analyzed sleep state analysis information of the user.

Meanwhile, the effects of the present invention are not limited to those mentioned above, and unmentioned other technical effects will be clearly understood by those skilled in the art from the following descriptions.

### BRIEF DESCRIPTION OF THE DRAWINGS

FIG. 1 is a view for explaining a system for providing sleep state analysis information and an improvement guide according to an embodiment of the present invention.

FIG. 2 is a view for more specifically explaining the memory unit of a system for providing sleep state analysis information and an improvement guide according to an embodiment of the present invention.

FIG. 3 is a view for explaining a system for providing sleep state analysis information and an improvement guide according to another embodiment of the present invention.

FIG. 4 is a view for explaining a method of providing sleep state analysis information and an improvement guide according to an embodiment of the present invention.

FIG. 5 is a view for explaining a method of providing sleep state analysis information according to an embodiment of the present invention.

FIG. 6 is a view for more specifically explaining a method of providing sleep state analysis information according to an embodiment of the present invention.

FIG. 7 is a view for explaining a method of providing sleep state analysis information according to this embodiment of the present invention.

FIG. 8 is a view for more specifically explaining a method of providing sleep state analysis information according to this embodiment of the present invention.

FIG. 9 is a view for explaining a method of providing sleep state analysis information and an improvement guide according to another embodiment of the present invention.

FIG. 10 is a view for explaining a method of providing sleep state analysis information and an improvement guide according to still another embodiment of the present invention.

FIG. 11 is a view showing sleep types produced by a system according to an embodiment of the present invention.

### BEST MODE FOR CARRYING OUT THE INVENTION

Details of the objects and technical configurations of the present invention and effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software components that perform the same function.

In addition, the expressions including certain components are expressions of “open type” and only refer to existence of corresponding components, and should not be construed as excluding additional components. Furthermore, when a certain component is referred to as being “connected” or “coupled” to another component, it may be directly connected or coupled to another component, but it should be understood that other components may exist in between.

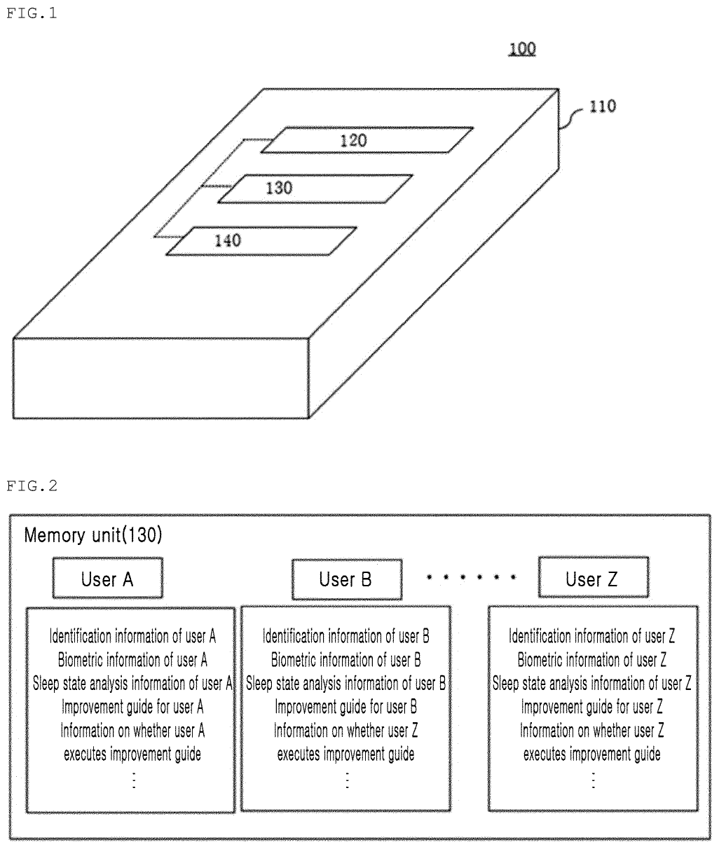

FIG. 1 is a view for explaining a system for providing sleep state analysis information and an improvement guide according to an embodiment of the present invention.

Referring to FIG. 1, a system 100 for providing sleep state analysis information and an improvement guide according to the present invention includes at least one layer unit 110 having a plurality of components, a detection unit 120 provided in the layer unit 110 to detect biometric information of a user, a memory unit 130 for storing the biometric information of the user detected by the detection unit 120, and an analysis unit 140 for analyzing a sleep state of the user on the basis of the biometric information of the user stored in the memory unit 130, and generating sleep state analysis information of the user.

The layer unit 110 may be understood as a member having an accommodation space in which the components described below can be arranged, and there is no limitation on the material or shape of the layer unit 110 as far as it has an accommodation space. The layer unit 110 may be a space where a user sleeps, for example, a mattress, or may be any one of a plurality of surfaces constituting the mattress. In addition, the layer unit 110 may be a mat that can be placed on the mattress, and further, may be a member made of a wood or metal material rather than a surface of a fiber material. As described, when the layer unit 110 has a predetermined accommodation space, there is no limitation on the material or shape. However, in this detailed description, it will be described on the assumption that the layer unit 110 is a mattress to help understanding of the invention.

The detection unit 120 is provided in the layer unit 110 to perform a function of detecting biometric information of the user. The detection unit 120 may be provided in various forms and locations, such as a form that can be attached to and detached from the surface or interior of the layer unit, or various wearable devices that can be directly/indirectly attached to and detached from the body of the user, and as long as it may perform a function of detecting biometric information of the user without disturbing the sleep of the user while the user sleeps, the form, type, location, number, arrangement, and the like thereof are not limited to the embodiments of the present invention.

In addition, the detection unit 120 may include at least one sensor among various types of sensors, for example, a pressure sensor, a vibration sensor, a position sensor, an acoustic sensor, an infrared sensor, a motion sensor, a face recognition sensor, a biometric recognition sensor, and a fingerprint recognition sensor. Specifically, the detection unit 120 may detect the weight or position information of the user through the pressure sensor, or acquire state information such as a heart rate, breathing state, movement state, and the like by detecting a vibration signal of the user through the vibration sensor. In addition, the detection unit 120 may recognize the voice of the user or detect snoring sounds through the acoustic sensor, or detect the gesture of the user through the motion sensor and receive movement information.

As described above, the detection unit 120 of the present invention is a component for detecting various types of biometric information of the user located on the layer unit 110, and adjusting the type, number, layout, and the like of sensors used to detect more precise and specific biometric information of a user may not be limited to the embodiments of the present invention. That is, the detection unit 120 of the present invention may include these various types of sensors in a form combining one or more sensors, and the biometric information of the user detected through the detection unit 120 may be transmitted to the memory unit 130 of the system 100 for providing sleep state analysis information and an improvement guide of the present invention through a communication unit (not shown).

The memory unit 130 is a component for storing the biometric information of the user detected by the detection unit 120, and may be provided to be embedded in the system 100 for providing sleep state analysis information and an improvement guide of the present invention, or may be an external device connected to the system 100 for providing sleep state analysis information and an improvement guide through a wired or wireless communication method.

Here, the biometric information of the user detected by the detection unit 120 may be stored in the memory unit 130 to be mapped to each of a plurality of users. For example, physical information such as the height, weight, and the like of user A or identification information that can distinguish a plurality of users from each other may be stored in the memory unit 130 to be mapped to each user, and collected biometric information of user A, such as face information, voice information, heart rate information, and the like, may be stored to be mapped to each user. The memory unit 130 is shown in FIG. 2 in detail, and FIG. 2 will be described below.

The analysis unit 140 analyzes the sleep state of a user on the basis of the biometric information of the user stored in the memory unit 130, and generates sleep state analysis information of the user. The analysis unit 140 may also be understood as a central processing unit, in which a predetermined data analysis and calculation process is performed. The central processing unit may also be referred to as a controller, a microcontroller, a microprocessor, a microcomputer, or the like. In addition, the central processing unit may be implemented by hardware, firmware, software, or a combination thereof. In the case of implementing using hardware, the central processing unit may be configured as an application specific integrated circuit (ASIC), a digital signal processor (DSP), a digital signal processing device (DSPD), a programmable logic device (PLD), a field programmable gate array (FPGA), or the like. In the case of implementing using firmware or software, the firmware or software may be configured to include modules, procedures, functions, or the like that perform the functions or operations described above. In the present invention, embodiments of the implementation form and data processing method of the analysis unit 140 cannot be interpreted in a limited way.

The analysis unit 140 analyzes at least one piece of information among the biometric information of the user stored in the memory unit 130, and generates sleep state analysis information of the user. More specifically, the sleep state of the user can be analyzed on the basis of the heart rate information (e.g., heart rate variability, etc.), respiration rate information (e.g., respiratory rate variability, etc.), pulse rate information, movement information, sound information, and the like of the user stored in the memory unit 130.

For example, the analysis unit 140 of the present invention may analyze state information, such as a lying down state, turned-over state, and getting-up state (such as getting out of the mattress, etc.) of the user through the information detected by the pressure sensor of the detection unit 120, analyze the heart rate information, respiration rate information, movement information, and the like of the user on through the information detected by the vibration sensor of the detection unit 120, and analyze sound information such as snoring or the like by detecting various sounds of the user through the acoustic sensor of the detection unit 120. Through this, the analysis unit 140 of the present invention may analyze the sleep state of the user, such as the heart rate variability, respiration rate variability, whether or not the user is awake (getting out of the mattress), and the like on the basis of various types of information of the user that have been analyzed.

In addition, the analysis unit 140 may analyze the sleep state of the user step by step, and when analyzing sleep stages, various parameters such as Total recording time (TRT), Total sleep time (TST), Time in bed (TIB), Sleep latency (SL), REM sleep latency (RL), Wake after sleep onset (WASO), Sleep efficiency (SE), duration of each sleep stage, percentage of each sleep stage, and the like may be utilized. Hereinafter, methods for actually calculating some parameters will be described.

Equations 1 and 2 are equations for calculating SE and SEw according to an embodiment of the present invention.

$$\begin{matrix}SE = \frac{T S T}{TIB} \\ TST = ( Sleep end time - Sleep start time ) - ( Sum of awake time ) \\ TIB = Sleep end time - Sleep start time\end{matrix} \qquad [ Equation 1 ]$$

Meanwhile, equation 2 is for calculating a weighted sleep efficiency (SEw) according to an embodiment of the present invention. The efficiency of human sleep can be evaluated by objective factors and subjective factors, and when SE introduced above in equation 1 is a parameter that reflects only the objective factors, equation 2 shown below may be a parameter that reflects even the subjective factors.

$$\begin{matrix}S {E}_{W} = \frac{T S {T}_{W}}{( T S T + {b}_{1} \cdot {WT}_{1} + {b}_{2} \cdot {WT}_{2} + {b}_{3} \cdot {WT}_{3} )} \\ TS {T}_{W} = a 1 \cdot [ Deep Sleep Duration ] + a 2 \cdot [ Light Sleep Duration ] + a 3 \cdot [ REM Sleep Duration ]\end{matrix} \qquad [ Equation 2 ]$$

In equation 2, [Deep Sleep Duration] means a time when a sleeper is in deep sleep, [Light Sleep Duration] means a time when the sleeper is in light sleep, and [REM Sleep Duration] means a time when the sleeper is in REM sleep. In addition, al to a3and b1 to b3 are weight values that can be arbitrarily set by the designer or system operator, WT₁ is a sum of time periods when the sleeper is awake for a predetermined period of time (e.g., minutes) after falling asleep, WT₂ is a sum of time periods when the sleeper is awake in a section excluding a predetermined period of time (e.g., minutes) after falling asleep and a predetermined period of time (e.g., minutes) before waking up, and WT₃ is a sum of time periods when the sleeper is awake for a predetermined period of time (e.g., minutes) immediately before waking up. The parameters included in the denominator are for reflecting the subjective factors related to the sleep of the sleeper. When a person is awake for a long period of time during the hypnagogia or before waking up, the person generally feels that he or she did not sleep well, and they are parameters for reflecting the subjective factors. In addition, for reference, deep sleep, light sleep, and REM sleep may be classified by predefined criteria (criteria based on biometric information that can be obtained from the sleeper, such as heart rate variability and respiratory rate variability).

As described above, the present invention may calculate a weighted sleep efficiency (SEw) by changing the weight in each sleep stage and applying an equation that changes the weight to the sleep time in each situation.

Meanwhile, equation 3 is for calculating a sleep score according to an embodiment of the present invention.

$$\begin{matrix}Sleep Score ( \% ) = {SE}_{W} \times 100 + c 1 \cdot min ( POSITIVE , A ) - c 2 \cdot min ( NEGATIVE , B ) + c 3 \cdot Subjective Sleep Score [ - 5 , 5 ] \\ Sleep Score = max ( min ( Sleep Score , 100 ) , 0 )\end{matrix} \qquad [ Equation 3 ]$$

In equation 3, the Subjective Sleep Score may be a subjective sleep satisfaction score measured by receiving an input for a questionnaire from a sleeper (user), and this may be converted into a value between −5 and 5 points.

In addition, when the Sleep Score in equation 3 has a value greater than 90, the sleep score may be evaluated as “good”, when it has a value greater than 70 and smaller than or equal to 90, the sleep score may be evaluated as “average”, and when it has a value smaller than or equal to 70, the sleep score may be evaluated as “bad”.

Meanwhile, the POSITIVE variable in equation 3 is for reflecting factors having a positive effect on the sleep score. According to an embodiment of the present invention, when the calculated SE (sleep efficiency) has a value greater than d₁, the analysis unit 140 may add (SE−d₁)*d₂ to the variable of ‘POSITIVE’. For reference, d₁ represents a reference point, and d₂ represents a weight value. The reference point of di is based on the premise that a predetermined value is assigned for normal sleep, good sleep, and the like, and the weight value may be set randomly. (For example: d₁=0.9, d₂=20)

An example of reflecting the POSITIVE variable is a case where a sleeper maintains a regular sleep state. For example, when the number of days in which the sleeper sleeps consistently during the last one week is L (e.g., 3) or more, the analysis unit 140 may add the number of days as a variable of ‘POSITIVE’. For example, the analysis unit 140 may determine whether the sleeper falls asleep in a predetermined time zone on the basis of whether the abs (time of falling asleep−average time of falling asleep for M (e.g., 7) days) has a value smaller than α (e.g., 30) minutes, and when the sleeper falls asleep in a predetermined time zone, the number of times may be determined as the number of days in which the sleeper falls asleep consistently.

In addition, when the number of days in which the sleeper wakes up consistently during the last one week is N (e.g., 3) or more times, the analysis unit 140 may add the number of days to the variable of ‘POSITIVE’. For example, the analysis unit 140 may determine whether the sleeper wakes up in a predetermined time zone on the basis of whether the abs (wake-up time−wake-up time for O (e.g., 7) days) has a value smaller than β (e.g., 30) minutes, and when the sleeper wakes up in a predetermined time zone, the number of times may be determined as the number of days in which the sleeper wakes up consistently.

Meanwhile, the NEGATIVE variable in equation 3 is for reflecting factors having a negative effect on the sleep score.

According to an embodiment of the present invention, when the TST is smaller than the average sleep time of each gender or age group as much as 1SD (standard deviation; a predetermined value) or more, the analysis unit 140 may add min (abs (TST−MEAN)/SD+1), el) to the variable of ‘NEGATIVE’. The value of el may be, for example, 5.

On the other hand, when the average sleep time (AST) for the last N (e.g., 2) days is smaller than the average sleep time of each gender/age group as much as 1SD or more, the analysis unit 140 may add min (abs ((AST−MEAN)/SD+1), e2) to the variable of ‘NEGATIVE’. The value of e2 may be, for example, 3.

On the other hand, when the SE is smaller than the AST, the analysis unit 140 may add abs (SE−e3)*e4 to the variable of ‘NEGATIVE’. The value of e3 may be, for example, 0.85, and the value of e4 may be, for example, 10.

On the other hand, when the sleep latency (SL) is greater than the average sleep latency of each gender/age group as much as 1SD or more, the analysis unit 140 may add min (abs ((SL-MEAN)/SD−1), e5) to the variable of ‘NEGATIVE’. Here, the sleep latency may represent the time taken from lying down in bed to falling asleep. The value of e5 may be, for example, 5.

On the other hand, when the persistent sleep latency (PSL) is greater than the SL, the analysis unit 140 may add min ((PSL-SL)*e6, e7) to the variable of ‘NEGATIVE’. At this point, the value of e6 may be, for example, 0.1, the value of e7 may be 5, and the PSL may be defined as a time taken to persist sleep for 10 minutes or more continuously after lying down in bed.

Meanwhile, according to another embodiment of the present invention, when the REM Sleep Latency (RSL) is greater than the average of each age group as much as 1SD or more, the analysis unit 140 may add min (abs ((RSL-MEAN)/SD−1), f1) to the variable of ‘NEGATIVE’. In this case, the value of f1 may be, for example, 5.

On the other hand, when the RSL is smaller than 15 minutes or there is no REM, the analysis unit 140 may add f2 to the variable of ‘NEGATIVE’. Here, the value of f2 may be, for example, 5, and the RSL may be defined as a time taken from SL to occurrence of REM represented in minutes.

On the other hand, in the case where % REM (the proportion occupied by the REM sleep) is out of the range of 15 to 30%, the analysis unit 140 may i) add min ((15−% REM)/f3, f4) to the variable of ‘NEGATIVE’ when % REM is smaller than 15%, or ii) add min ((% REM−30)/f3, f4) to the variable of ‘NEGATIVE’ when % REM is smaller than 30%. Here, % REM is a value representing the proportion (%) occupied by REM in the entire sleep, and f3 may be 5, and f4 may be 3.

On the other hand, when % DEEP is smaller than the average of each gender/age group as much as 1SD or more, the analysis unit 140 may add min ((% DEEP31 MEAN)/SD+1), f5) to the variable of ‘NEGATIVE’. Here, % DEEP is a value representing the proportion (%) of DEEP in entire total sleep, and f5 may be 5.

On the other hand, when the sleep cycle (SC) is smaller than f6 (e.g., 3), the analysis unit 140 may add min (abs(SC−3)* f7) to the variable of ‘NEGATIVE’ (f7 is, for example, 2). Here, the sleep cycle may represent the number of times of sequentially changing the sleep stages of wake, light sleep, deep sleep, light sleep, and REM during a sleep.

On the other hand, in the case where the Apnea-Hypopnea Index (AHI) is greater than 5, the analysis unit 140 may add a factor to the variable of ‘NEGATIVE’ by applying 10 to f8 when the AHI is 30 or higher, applying 6 to f8 when the AHI is 15 or higher, and applying 3 to f8 when the AHI is 5 or higher. Here, the AHI is a value representing the number of times (count/hour) that the apnea and hypopnea occur per hour. Thereafter, the analysis unit 140 may calculate a physical recovery index for evaluating information on the recovery obtained by the sleep. It should be noted that the f values may vary according to the intention of the designer or operator.

Meanwhile, according to another embodiment of the present invention, the analysis unit 140 may use the heart rate measured during the sleep to calculate an autonomic nervous system activity index, which is a heart rate variability index indicating the sympathetic nerve activity and the parasympathetic nerve activity. For example, the analysis unit 140 may use the index to estimate a degree of refreshment after waking up (refreshing score) and a degree of fatigue the day after sleep (fatigue score).

According to an embodiment of the present invention, the analysis unit 140 may receive a degree of refreshment after waking up from the analysis targets. For example, the degree of refreshment may be expressed as a value closer to 0 when the degree of fatigue is not improved at all at 7 PM on that day, and a value closer to 100 when the degree of fatigue is improved greatly. Thereafter, the analysis unit 140 may determine a physical recovery index as a weighted sum of the estimated ‘degree of refreshment after waking up’ and ‘degree of fatigue the day after sleep’.

Equation 4 is for calculating the physical recovery index (ADJ Score) according to an embodiment of the present invention.

$$\begin{matrix}ADJ Score = ( p 1 \cdot refreshing score + ( 100 - p 2 \cdot fatigue score ) ) / 2 = f ( \frac{H F}{L F} , nHF , nLF ) \\ = a \cdot H F / LF + b \cdot nHF + c \cdot nLF + d \cdot H F / LF \cdot nLF + e \cdot H F / LF \cdot nHF + f \cdot nHF \cdot nLF + g \\ nHF = \frac{H F}{L F + H F} \\ nLF = \frac{L F}{L F + H F}\end{matrix} \qquad [ Equation 4 ]$$

According to an embodiment of the present invention, as input variables for estimating the degree of refreshment after waking up and the degree of fatigue the next day, the analysis unit 140 may use: (i) the proportion of each heart rate variability (HRV) index, which reflects autonomic nervous system activity, exceeding a threshold value during sleep, (ii) HRV indices calculated every 30 seconds, and (iii) the proportion of specific indices (e.g., HF/LF ratio, nHF, nLF) exceeding a threshold value during the entire sleep period.

According to an embodiment of the present invention, the analysis unit 140 may confirm the estimated degree of refreshment after waking up and the degree of fatigue the day after sleep as result variables, and perform modeling of generating a model for each of the degree of refreshment after waking up and the degree of fatigue the day after sleep by applying the result variables to an nth-order polynomial of input variables.

Meanwhile, according to another embodiment of the present invention, the detection unit 120 may include an unconstrained sensor, and the analysis unit 140 may calculate a sleep regularity index (SRI) and a chronotype index (MSFsc) using long-term sleep information detected through the unconstrained sensor, and derive a sleep type by combining the indexes. For example, it is known that when the sleep regularity index is divided into three types and the chronotype is divided into five types, 15 types of chronotypes can be derived. The SRI types may be subdivided into, for example, ‘good’ when the SRI value is larger than or equal to 84, ‘normal’ when the SRI value is smaller than 84 and larger than or equal to 60.8, and ‘bad’ when the SRI value is smaller than 60.8.

The MSFsc type may be divided into units of epochs elapsed time from midnight. In this case, 1 epoch may mean 30 For example, 366 epochs may represent 3:03 AM elapsed seconds. 183 minutes from midnight, and 400 epochs may represent 3:20 AM elapsed 200 minutes from midnight.

The MSFsc type may be subdivided into an extreme morning type when the epoch value is smaller than 366, a moderate type when the epoch value is larger than or equal to 366 and smaller than 400, an intermediate type when the epoch value is larger than or equal to 400 and smaller than 477, a moderate evening type when the epoch value is larger than or equal to 477 and smaller than 507, and an extreme evening type when the epoch value is larger than or equal to 507.

The analysis unit 140 may convert the calculated sleep score into a score between a minimum of 0 and a maximum of 100. For example, the analysis unit 140 may determine the state of short-term sleep as ‘good’ when the converted sleep score exceeds 90 points, ‘average’ when the sleep score exceeds 70 points and smaller than or equal to 90 points, and ‘bad’ when the sleep score is smaller than or equal to 70 points.

In addition, the analysis unit 140 of the present invention may analyze the biometric information of the user stored in the memory unit 130 in a short term, calculate a result thereof as a numerical index, and generate sleep state analysis information of the user by including the numerical index, or/and may analyze the biometric information of the user stored in the memory unit 130 in a long term, determine a sleep state type of the user on the basis of the difference in the sleep state during the long-term analysis period, and generate sleep state analysis information of the user by including the sleep state type. For reference, the short term or short-term data mentioned in this detailed description means 1 day or data obtained in 1 day, and the long term or long-term data means 10 days or more or data obtained during a period of 10 days or more.

The sleep state analysis information of the user generated in the analysis unit 140 may be stored in the memory unit 130. Hereinafter, the memory unit 130 of the present invention will be described in more detail with reference to FIG. 2.

FIG. 2 is a view for more specifically explaining the memory unit 130 of a system for providing sleep state analysis information and an improvement guide according to an embodiment of the present invention, and as shown in FIG. 2, the memory unit 130 may store biometric information and sleep state analysis information of a user, in addition to identification information that can identify the user. In particular, according to the present invention, when the layer unit 110 is used by a plurality of users, information on each of the plurality of users may be stored in the memory unit 130 to be mapped to the biometric information and sleep state analysis information. For example, when information on a plurality of user is stored in the memory unit 130, identification information that can distinguish user A and biometric information of user A may be stored to be mapped to the user information, and sleep state analysis information of user A analyzed on the basis of the stored biometric information may be stored to be mapped together.

Here, according to the present invention, as the biometric information, sleep state analysis information, and the like are stored in the memory unit 130 to be mapped to each of a plurality of users, together with the identification information of the user, although the system 100 for providing sleep state analysis information and an improvement guide according to the present invention is commonly used by a plurality of users, detection of biometric information, analysis of an sleep state, and provision of an improvement guide are possible for each specific user accurately.

In other words, according to the present invention, identification information, biometric information, and sleep state analysis information mapped to each user may be stored in the memory unit 130, and based on the biometric information of the user, an optimal improvement guide that can change the sleep state of the user may be generated for one user or/and for each user of a plurality of users.

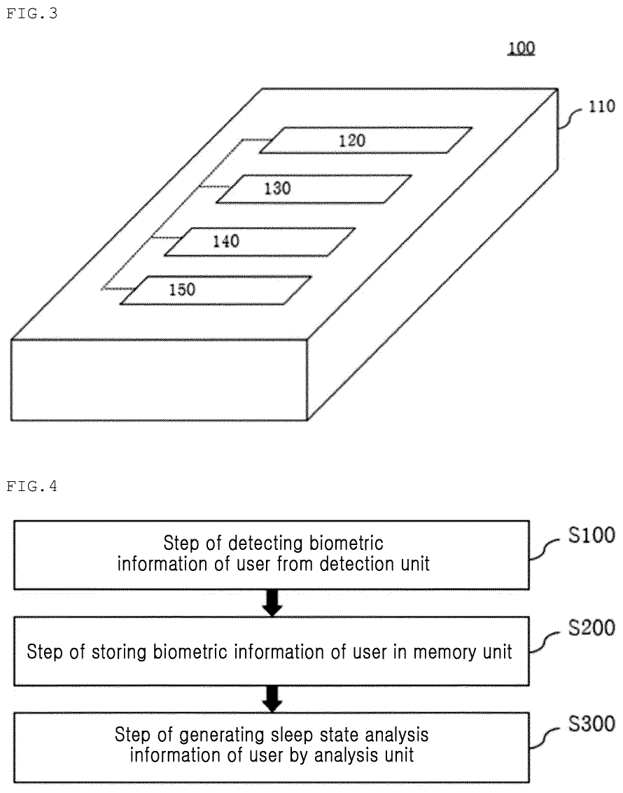

Hereinafter, a system for providing sleep state analysis information and an improvement guide according to another embodiment of the present invention will be described with reference to FIG. 3.

FIG. 3 is a view for explaining a system for providing sleep state analysis information and an improvement guide according to another embodiment of the present invention, and as shown in FIG. 3, the analysis unit 140 may generate sleep state analysis information of the user on the basis of the biometric information of the user stored in the memory unit 130, and also generate an improvement guide that can change the sleep state of the user on the basis of the biometric information and sleep state analysis information of the user stored in the memory unit 130.

The sleep state improvement guide of the user generated by the analysis unit 140 may be provided to the user in various types, and in addition, according to another embodiment of the present invention, a feedback receiving unit 150 for monitoring information on whether the user executes the improvement guide generated by the analysis unit 140 may be further included. The feedback receiving unit 150 is a component that can monitor the behavior information of the user and/or the sleep state information of the user and may be provided inside or outside the layer unit 110, and in addition, may be a component included in the detection unit or a component the same as the detection unit.

For example, when the numerical index of the sleep state of the user is calculated to be 50 points and is desired to be improved to 70 points or higher, or/and when the sleep state of the user is analyzed as Type I sleep state and is desired to be changed to Type II sleep state, the analysis unit 140 may provide an improvement guide needed to change the sleep state of the user, such as an improvement guide including behaviors of daily life, behaviors to be performed before sleeping, and e like. Furthermore, through the feedback receiving unit 150, it is possible to monitor whether the user properly performs the behaviors according to the provided improvement guide, and whether the sleep state of the user is improved by a behavioral pattern, environment, and the like suitable for changing to Type II.

As described above, the system for providing sleep state analysis information and an improvement guide according to the present invention detects biometric information of the user, analyzes the sleep state of the user on the basis of the detected biometric information, calculates a result thereof as a numerical index by analyzing the sleep state in a short term, or/and determines a sleep state type by analyzing the sleep state in a long term, and provide sleep state analysis information of the user.

Through the present invention as described above, as the user may more intuitively recognize details of his or her own sleep state through the sleep state analysis information provided in the form of a numerical index and/or a sleep state type, the user may quickly and easily understand his or her sleep state without help of an expert, and may also easily predict the effect of his or her sleep state on the daily life, and through this, the user may prepare in advance for problems in his or her daily life that may occur due to degradation in the quality of the sleep state.

In addition, the present invention may provide an improvement guide including specific behaviors that can change the sleep state when the user desires to change his or her sleep state, and through this, there is an advantage in that the user may improve the sleep state to an optimal sleep state type suitable for him or her simply by following the improvement guide provided by the system of the present invention.

In addition, the present invention may obtain an effect of providing a better improvement guide that can change the sleep state type of a user more effectively by receiving feedback on whether the user executes the behavior guidelines of the improvement guide provided to the user and monitoring how much the change in the behavior of the user actually has affected the change in the sleep state type of the user.

Hereinafter, a method of providing sleep state analysis information and an improvement guide according to an embodiment of the present invention will be described with reference to FIG. 4.

FIG. 4 is a view for explaining a method of providing sleep state analysis information and an improvement guide according to an embodiment of the present invention.

Referring to FIG. 4, a method of providing sleep state analysis information and an improvement guide, which is performed in a system for providing sleep state analysis information and an improvement guide including a layer unit, a detection unit, a memory unit, and an analysis unit, includes a step of detecting biometric information of a user from the detection unit provided in the layer unit (S100), a step of storing the biometric information of the user detected by the detection unit in the memory unit (S200), and a step of analyzing a sleep state of the user on the basis of the biometric information of the user stored in the memory unit, and generating sleep state analysis information of the user, by the analysis unit (S300).

The layer unit may be a space where a user sleeps, for example, a mattress, or may be any one of a plurality of surfaces constituting the mattress. In addition, the layer unit may be a mat that can be placed on the mattress, and further, may be a member made of a wood or metal material rather than a surface of a fiber material. As described, when the layer unit has a predetermined accommodation space, there is no limitation on the material or shape. However, in this detailed description, it will be described on the assumption that the layer unit is a mattress to help understanding of the invention.

Step S100 of collecting biometric information of a user from the detection unit is performed by the detection unit provided in the layer unit, and the detection unit includes at least one sensor among various types of sensors, for example, a pressure sensor, a vibration sensor, a position sensor, an acoustic sensor, an infrared sensor, a motion sensor, a face recognition sensor, a biometric recognition sensor, and a fingerprint recognition sensor, and adjusting the type, number, layout, and the like of sensors used to detect more precise and specific biometric information of a user may not be limited to the embodiments of the present invention.

Specifically, step S100 may be performed in various ways, such as detecting the weight or position information of the user through the pressure sensor of the detection unit, detecting state information such as a heart rate, breathing state, movement state, and the like by detecting a vibration signal of the user through the vibration sensor, recognizing the voice of the user or detecting snoring sounds through the acoustic sensor, or detecting the gesture of the user through the motion sensor and receiving movement information.

Next, the step of storing the biometric information of the user detected by the detection unit in the memory unit (S200) is performed. Step S200 may be performed in a way that the biometric information of the user collected by the detection unit is stored to be mapped to each of a plurality of users. Through this, even when a plurality of users commonly uses the mattress, the biometric information of the user may be stored to be mapped to the identification information that can distinguish each user. (See FIG. 2)

Then, the analysis unit performs step S300 of analyzing a sleep state of the user on the basis of the biometric information of the user stored in the memory unit, and generating sleep state analysis information of the user, by the analysis unit. Here, step S300 may be performed by the analysis unit in a way of analyzing at least one piece of information among the biometric information of the user stored in the memory unit, and generating sleep state analysis information of the user. Specifically, the sleep state of the user may be analyzed through the heart rate information (e.g., heart rate variability, etc.), respiration rate information (e.g., respiratory rate variability, etc.), pulse rate information, movement information, sound information, and the like of the user.

In other words, according to the present invention, the state information, such as a lying down state, turned-over state, and getting-up state (such as getting out of the mattress, etc.) of the user, can be analyzed through the information detected by the pressure sensor of the detection unit, and the heart rate information, respiration rate information, movement information, and the like of the user can be analyzed through the information detected by the vibration sensor of the detection unit, and sound information such as snoring or the like can be analyzed by detecting various sounds of the user through the acoustic sensor of the detection unit. Through this, at step S300 of the present invention, the analysis unit may analyze the sleep state of the user, such as the heart rate variability, respiration rate variability, whether or not the user is awake (getting out of the mattress), and the like on the basis of the biometric information of the user stored in the memory unit.

In addition, step S300 may also be performed in a way of analyzing the sleep state of the user step in steps by the analysis unit, and when analyzing sleep stages, various parameters such as Total recording time (TRT), Total sleep time (TST), Sleep latency (SL), REM sleep latency (RL), Wake after sleep onset (WASO), Sleep efficiency (SE), duration of each sleep stage, percentage of each sleep stage, and the like may be utilized.

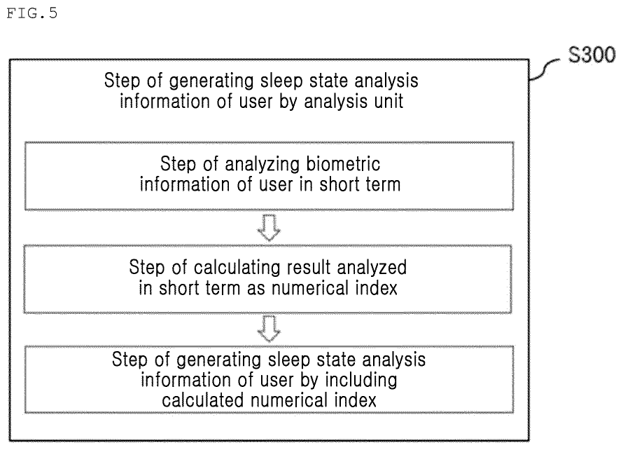

Hereinafter, the process of generating the sleep state analysis information of the user according to the present invention will be described in more detail with reference to FIGS. 5 to 9.

FIG. 5 is a view for explaining a method of providing sleep state analysis information according to an embodiment of the present invention, and as shown in FIG. 5, the step of generating sleep state analysis information of the user by the analysis unit (S300) may be performed in a way of analyzing the biometric information of the user stored in the memory unit in a short term, calculating a result thereof as a numerical index, and generating sleep state analysis information of the user by including the numerical index.

Specifically, the step of generating sleep state analysis information of the user (S300) according to an embodiment of the present invention may be performed to include a step of analyzing the biometric information of the user stored in the memory unit in a short term, a step of calculating a result of analyzing in a short term as a numerical index, and a step of generating the sleep state analysis information of the user by including the calculated numerical index.

That is, according to the present invention, as the biometric information of the user is analyzed in a short term, various types of accurately analyzed sleep state analysis information may be provided to the user every day, and through this, the present invention is advantageous in that a user may establish every day the life, goal, plan, and the like of the next day more productively and effectively on the basis of the sleep state analysis information of the user himself or herself.

In addition, the present invention may provide the analyzed sleep state analysis information of the user by summarizing it as a single numerical index, i.e., calculating the sleep state analysis information as a score, rather than providing it as a lengthy explanatory material.

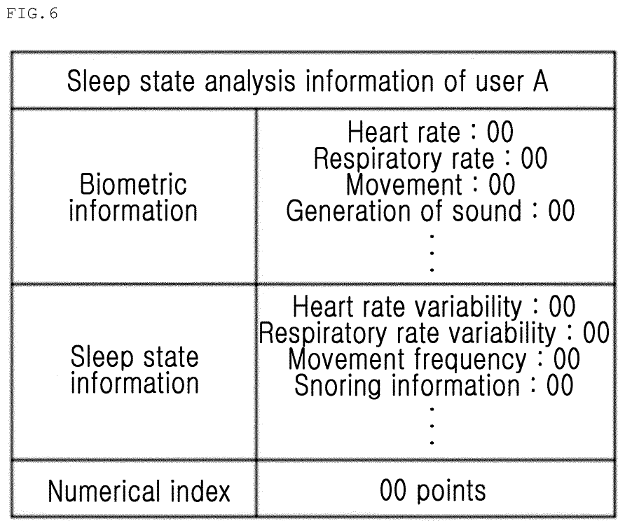

FIG. 6 is a view for more specifically explaining a method of providing sleep state analysis information according to an embodiment of the present invention, and as shown in FIG. 6, sleep state information (e.g., heart rate variability, respiratory rate variability, movement frequency, snoring information, etc.) of user A can be grasped specifically on the basis of the biometric information (e.g., heart rate, respiratory rate, movement, generation of sound, etc.) of user A, and this may be calculated and provided as a single numerical index.

Analysis of the sleep state information and calculation of the numerical index like this may utilize both subjective information and/or objective information in various ways. For example, when the sleep state information of a user is calculated as a numerical index, a weighted sleep efficiency (WSE) can be calculated according to an equation known in advance or defined according to the present invention. The equation of the present invention used to calculate the numerical index may be a single equation or a combination of at least two equations, and the embodiments of the present invention cannot be limited to the number of combinations of these equations and combination methods thereof.

In addition, these equations may be equations that utilize at least one pieces of information among various types of subjective information that can be recognized through experience, for example, information such that when a user does not fall asleep and stays awake for a long time while lying down in the early stage of the sleep section, this remains well in the memory of the user so that the user thinks that he or she does not sleep well, information such that when a user frequently wakes up from the sleep before waking up in the late stage of the sleep section, this remains well in the memory of the user so that the user thinks that he or she does not sleep well, and information such that when there is a plurality of sections in the sleep section, during which the user stays awake for a predetermined period of time or more, this remains well in the memory of the user so that the user thinks that he or she does not sleep well. In addition, these equations may be equations that utilize objective information proven through a plurality of papers or research results, for example, information such that deep sleep is a sleep state that affects physical recovery, acceleration of growth hormone, and consolidation of declarative memory, and information such that deep sleep is a sleep state that affects mental recovery, consolidation of procedural memory, and the like.

Meanwhile, a sleep state is closely related to the daily life of a user. In particular, many research results are known to show that when the sleep state the day before is not good, it affects the daily life of the user on the next day, such as increase of accidents, decrease of attention, decrease of cognitive behaviors, and the like. As a test for evaluating the relationship between the sleep state and the daily life, there is, for example, an Epworth Sleepiness Scale (ESS) evaluation, and the like. In the present invention, it is also possible to analyze the effect of sleep on the daily life of a user through the sleep state analysis information and sleepiness self-evaluation of the user.

The sleepiness self-evaluation may be performed in a way of evaluating a level of sleepiness as a number by the user himself or herself when performing each activity described as an example, and then adding up evaluation scores. For example, when the user is not sleepy at all when performing an activity of sitting and reading a book, this may be evaluated as “0”, when the user is sleepy a little, this may be evaluated as “1”, when the user is quite sleepy, this may be evaluated as “2”, and when the user is very sleepy, this may be evaluated as “3”. In addition, the level of daytime sleepiness may be evaluated by adding up the evaluation scores of each level of sleepiness for various activities.

Here, according to the present invention, the effect of the sleep state of the user on daily life (e.g., criteria for daytime sleepiness) may be analyzed on the basis of the sleepiness self-evaluation history of the user by performing sleepiness self-evaluation after the sleep state of the user is analyzed, and analyzing the sleep state analysis information and the sleepiness self-evaluation result of the user together. For example, when the sleep state analysis result of user A is calculated to be 75 points, the sleepiness self-evaluation index of the user may be predicted to be approximately 11 points (a slight sleepy state in daytime), and as the predicted information is included in the sleep state analysis information and provided to the user, the user may be provided with how the sleep state may affect the daily life of the user, in addition to the information on the sleep state.

That is, as the present invention estimates self-evaluated sleepiness by using information related to the sleep state, derives a result thereof, and in particular, uses a regression model based on a large amount of data accumulated over several times, a user may be provided with specific evaluation criteria for daytime sleepiness to determine how much such sleep state may affect the daily life of the user, together with information on the analysis of the sleep state.

In other words, the present invention may intuitively provide a result of analyzing the sleep state of a user by calculating the result as a numerical index, and provide a result of analyzing the effect of a sleep state on the daily life through sleepiness self-evaluation, and through this, the user may be provided with prediction information on how much the sleep state may cause sleepiness and fatigue in daily life, together with information on the sleep state of the user himself or herself. Therefore, the user may establish the life, goal, plan, and the like of the next day more productively and effectively on the basis of the sleep state analysis information of the user himself or herself.

Although an embodiment of utilizing the sleepiness self-evaluation as an index of how a sleep state of a user affects daily life has been illustrated and described above, the embodiment of the present invention is not limited thereto, and various evaluation indexes that can show the correlation between a sleep state and daily life of a user may be utilized as other embodiments of the present invention, and at least one of the evaluation indexes may be utilized in combination.

In addition, according to this embodiment of the present invention, information on the sleep state of a user can be accumulated, collected, and stored, and the sleep state of the user can be analyzed in a long term by utilizing the accumulatively stored information. That is, when a user sleeps using the layer unit of the system for providing sleep state analysis information and an improvement guide of the present invention several times or more, the biometric information and/or sleep state analysis information of the user can be accumulatively stored, and through this, long-term sleep state analysis information of the user can be grasped.

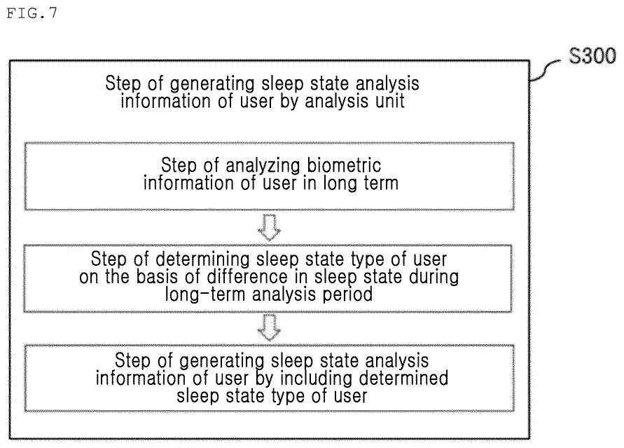

FIG. 7 is a view for explaining a method of providing sleep state analysis information according to this embodiment of the present invention, and as shown in FIG. 7, the step of generating sleep state analysis information of the user by the analysis unit (S300) may be performed in a way of analyzing the biometric information of the user stored in the memory unit in a long term, categorizing the sleep state within the long-term analysis period, and generating the sleep state analysis information of the user by including the sleep state.

Specifically, the step of generating sleep state analysis information of the user (S300) according to this embodiment of the present invention may be performed to include a step of analyzing the biometric information of the user stored in the memory unit in a long term, a step of determining a sleep state type of the user on the basis of the difference in the sleep state during the long-term analysis period, and a step of generating sleep state analysis information of the user by including the determined sleep state type of the user.

That is, according to the present invention, as the accumulatively stored biometric information and/or sleep state analysis information of the user is analyzed in a long term, the sleep state type of the user can be determined on the basis of the difference in the sleep state of the user, and for example, long-term sleep state analysis information, such as whether the sleep state of the user is regular or irregular or whether there is any difference between the sleep states of the user on weekdays and weekends, can be extracted. In addition, the present invention may also determine the sleep type of the user on the basis of the long-term sleep state analysis information.

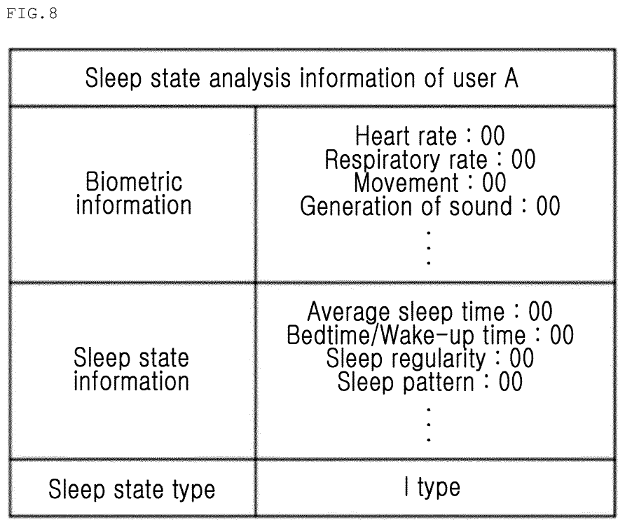

FIG. 8 is a view for more specifically explaining a method of providing sleep state analysis information according this an embodiment of the present invention, and as shown in FIG. 8, sleep state information (e.g., average sleep time, bedtime/wake-up time, sleep regularity, sleep pattern, etc.) of user A can be grasped specifically on the basis of the biometric information (e.g., heart rate, respiratory rate, movement, generation of sound, etc.) of user A, and this may be defined as various sleep state types and included in the long-term sleep state analysis information.

For example, the long-term sleep state analysis information may include an average sleep time (evaluation of whether the user gets enough sleep), bedtime/wake-up time (determining whether the sleep type of the user is an evening type or a morning type), regularity of weekday sleep (evaluation of whether the user gets regular sleep during weekdays), regularity of sleep patterns on weekdays and weekends (evaluation of whether the weekday and weekend sleep patterns of the user are regular, or whether there is a difference between the weekday and weekend sleep patterns, etc.) of the user. In addition, a time when the user is not on the mattress may be classified as a non-sleep activity time, and the daily life-sleep circadian rhythm of the user can be evaluated on the basis of the non-sleep activity time. In addition, in the process of generating the long-term sleep state analysis information in the present invention, the amount of activity, delayed sleep phase syndrome, and the like of the user may be evaluated, and in addition, graphs for evaluating sleep regularity may also be generated by identifying the user's behavior of lying down in bed, behavior of getting up from bed, and the like.

In the present invention, the long-term sleep state analysis information generated in this way may be clustered according to predetermined criteria, defined as one sleep state type, and provided to be included in the sleep state analysis information of the user. The sleep state categorization may be performed by a single technique or by combining at least two or more of various techniques, and the type, number, combination method, or/and criteria for defining the sleep state type, the number of sleep state types, the sleep state types, and the like of the categorization techniques cannot be limited to the embodiment of the present invention.

For example, in the present invention, a clustering technique may be used for sleep state categorization, and the clustering technique is one of machine learning techniques and means a process of clustering a plurality of data into a plurality of groups on the basis of similarity. When it is desired to analyze a large amount of accumulatively stored data during a long-term period and define a specific sleep state type as shown in this embodiment of the present invention, the clustering technique can be utilized effectively. However, the clustering technique is not necessarily used for sleep state categorization in the present invention.

As described above, as the present invention analyzes the sleep state of a user in a long term and provides an analysis result in a form including a sleep state type, the user may intuitively grasp his or her sleep state in a categorized form. In addition, another embodiment of the present invention may provide an improvement guide that can change the sleep state type of the user.

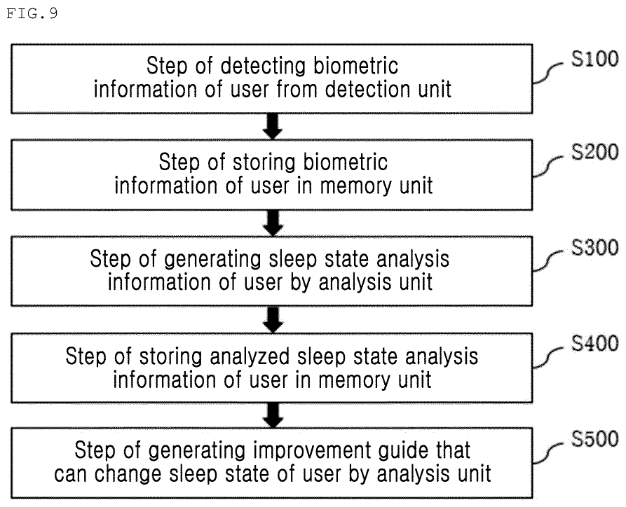

FIG. 9 is a view for explaining a method of providing sleep state analysis information and an improvement guide according to another embodiment of the present invention.

As shown in FIG. 9, after the step of detecting biometric information of a user from the detection unit provided in the layer unit (S100), the step of storing the biometric information of the user detected by the detection unit in the memory unit (S200), and the step of analyzing a sleep state of the user on the basis of the biometric information of the user stored in the memory unit, and generating sleep state analysis information of the user, by the analysis unit (S300), a step of storing the analyzed sleep state analysis information of the user in the memory unit (S400), and a step of generating an improvement guide that can change the sleep state of the user by the analysis unit (S500) may be further performed.

The step of storing the analyzed sleep state analysis information of the user in the memory unit (S400) may be performed to store the sleep state analysis information to be mapped to the identification information of the user, and in addition, in the case of a mattress commonly used by a plurality of users, the step may be performed to store the sleep state analysis information of the user to be mapped to each of the plurality of users. (See FIG. 2)

Next, the step of generating an improvement guide that can change the sleep state of the user by the analysis unit (S500) may be performed to generate an improvement guide for the user on the basis of the biometric information of the user and the sleep state analysis information of the user stored in the memory unit, and in addition, in the case of a mattress commonly used by a plurality of users, an improvement guide for each user may be generated to be mapped to each user on the basis of information stored in the memory unit (see FIG. 2).

Here, in the present invention, at the step of generating sleep state analysis information of the user by the analysis unit (S300), the sleep state analysis information of the user may be provided in a form including a numerical index converted into a score and/or a sleep state type, and the analysis unit of the present invention may generate an improvement guide that can change the sleep state of the user. For example, when the numerical index of the sleep state of the user is calculated to be 50 points and is desired to be improved to 70 points or higher, or/and when the sleep state of the user is analyzed as Type I sleep state and is desired to be changed to Type II sleep state, the analysis unit may generate an improvement guide needed to change the sleep state of the user, and the improvement guide may include, for example, behaviors of daily life, behaviors to be performed before sleeping, and the like.

The sleep state improvement guide generated at step S500 may be provided to the user in various ways, and furthermore, according to another embodiment of the present invention, a process of monitoring information on whether the user executes the improvement guide may be additionally performed.

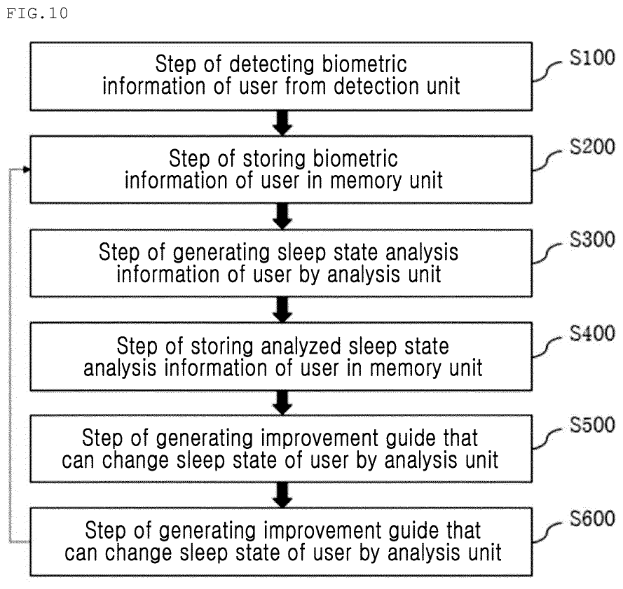

FIG. 10 is a view for explaining a method of providing sleep state analysis information and an improvement guide according to still another embodiment of the present invention, and referring to FIG. 10, after the step of generating an improvement guide that can change the sleep state of the user by the analysis unit (S500) is performed, a step of monitoring information on whether the user executes the improvement guide by the feedback receiving unit (S600) may be performed.

The feedback receiving unit is a component that can monitor the behavior information of the user and/or the sleep state information of the user and may be provided inside or outside the layer unit, and in addition, it may be a component included in the detection unit or a component the same as the detection unit. Step S600 may be performed in the feedback receiving unit in a way of monitoring whether the user executes well the behaviors included in the improvement guide, and accordingly whether the sleep state of the user is improved by a behavioral pattern, environment, and the like suitable for change.

In addition, the present invention may include, after the step of monitoring information on whether the user executes the improvement guide by the feedback receiving unit (S600), a process of returning to the step of storing a result of monitoring information on execution of the user in the memory unit (S200), and repeatedly performing the steps thereafter at least once.

That is, information on the result of executing the improvement guide for changing the sleep state of the user may be stored in the memory to be mapped to each user, together with the identification information of the user (see FIG. 2), and whether the process of analyzing the sleep state of the user is repeatedly performed after the improvement guide is executed and therefore the sleep state of the user has actually changed due to the improvement guide can be continuously observed, and through this, it can be utilized to generate an optimal improvement guide that can effectively change the sleep state of the user.

As described above, in the present invention, as a result of analyzing biometric information of a user in a short term and/or long term is calculated as a numerical index, or the sleep state analysis information is generated to include the details defined as a sleep state type, the user may more intuitively recognize his or her sleep state, and as criteria that specifically analyzes how much the sleep state affects the daily life of the user is provided, the user may predict his or her daily life affected by the sleep state and establish countermeasures.

In addition, as the present invention provides an improvement guide that can change the sleep state of a user on the basis of the sleep state analysis information of the user, it may help the user to have a sleep state most efficient: for himself/herself, and as information on the result of executing the improvement guide is accumulatively stored and utilized for analysis of the sleep state of the user and generation of a sleep state improvement guide, it may provide the user with an effect of sleeping better.

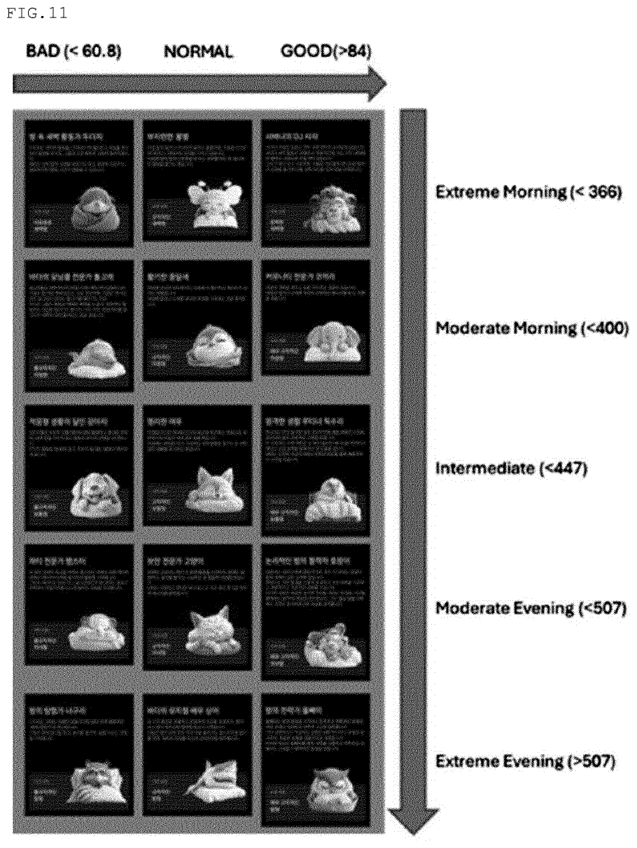

FIG. 11 is a view showing sleep types produced by a system according to an embodiment of the present invention.

Referring to FIG. 11, the sleep type may include 15 sleep types, which is a combination of three SRI types and five MSFsc types.

According to an embodiment of the present invention, the analysis unit 140 may determine an SRI type and an MSFsc type on the basis of data in bed for N weeks, and determine a sleep type. For example, the SRI type may include a type of ‘good’ when the SRI value is larger than or equal to 84, ‘normal’ when the SRI value is smaller than 84 and larger than or equal to 60.8, and ‘bad’ when the SRI value is smaller than 60.8. The MSFsc type may be subdivided into a type of extreme morning when the epoch value is smaller than 366, moderate when the epoch value is larger than or equal to 366 and smaller than 400, intermediate when the epoch value is larger than or equal to 400 and smaller than 477, moderate evening when the epoch value is larger than or equal to 477 and smaller than 507, and extreme evening when the epoch value is larger than or equal to 507.

According to an embodiment of the present invention, the sleep type is a dawn type, and may include a free dawn type combining an MSFsc type of ‘extreme morning’ with an SRI type of ‘bad’, a regular dawn type combining ‘extreme morning’ with ‘normal’, and a perfect dawn type combining ‘extreme morning’ with ‘good’. For reference, an animal corresponding to each type may be mapped to help intuitive recognition of the user, and a specific animal and a supplementary expression may be mapped together, and for example, the free dawn type may be mapped to an “underground dawn activist mole”, the regular dawn type may be mapped to an “industrious honeybee”, and the perfect dawn type may be mapped to an “DJ lion of the savanna”, and so on. Such mapping of animals and supplementary expressions may induce interest of a user, and may have an effect of enhancing convenience of the user by allowing the user to intuitively understand his or her sleep type.

According to an embodiment of the present invention, the sleep type is a regular morning type, and may include an irregular morning type (morning call expert dolphin in the ocean) combining an MSFsc type of ‘moderate’ with an SRI type of ‘bad’, a regular morning type (energetic lark) combining ‘moderate’ with ‘normal’, and a very regular morning type (community expert elephant) combining ‘moderate’ with ‘good’.

According to an embodiment of the present invention, the sleep type is a regular normal type, and may include an irregular normal type (adaptive life master puppy) combining an MSFsc type of ‘intermediate’ with an SRI type of ‘bad’, a regular normal type (clever fox) combining ‘intermediate’ with ‘normal’, and a very regular normal type (strict life routineer eagle) combining ‘intermediate’ with ‘good’.

According to an embodiment of the present invention, the sleep type is a regular evening type, and may include an irregular evening type (party expert hamster) combining an MSFsc type of ‘moderate evening’ with an SRI type of ‘bad’, a regular evening type (security expert cat) combining ‘moderate evening’ with ‘normal’, and a very regular evening type (logical night philosopher tiger) combining ‘moderate evening’ with ‘good’.

According to an embodiment of the present invention, the sleep type is a regular night type, and may include an irregular night type (night explorer raccoon) combining an MSFsc type of ‘extreme evening’ with an SRI type of ‘bad’, a regular night type (sea musical actor shark) combining ‘extreme evening’ with ‘normal’, and a very regular night type (night strategist owl) combining ‘extreme evening’ with ‘good’.

According to an embodiment of the present invention, the analysis unit 140 may provide an improvement guide for a sleep type on the basis of a determined sleep type. For example, when the sleep type determined for the user is ‘raccoon type’ and the user desires to change to a ‘fox type’, the analysis unit 140 may provide the user with an improvement guide including steps that make the bedtime and wake-up time of the user fall within the range of ‘fox type’.

According to an embodiment of the present invention, the analysis unit 140 may set a step for changing the SRI type. For example, in relation to the SRI type, the analysis unit 140 may set a range between 1 hour before or after a suggested time and 1 hour before or after the wake-up time as a first step for changing the bedtime, and may set a range between 40 minutes before or after the suggested time and 50 minutes before or after the wake-up time as a second step.

According to an embodiment of the present invention, in relation to the MSFsc type, the analysis unit 140 may set a step for changing the MSFsc type. For example, the analysis unit 140 may set the ranges of bedtime and wake-up time by setting 15 minutes before or after the current bedtime and setting 20 minutes before or after the wake-up time as a first step for changing the bedtime and wake-up time. In addition, the analysis unit 140 may set 30 minutes before or after the current bedtime and 20 minutes before or after the wake-up time as a second step.

According to an embodiment of the present invention, the analysis unit 140 may provide information that allows a user to follow the set steps as the improvement guide. For example, the analysis unit 140 may provide improvement guides for the SRI type and the MSFsc type simultaneously, or may sequentially provide an improvement guide for the SRI type first and provide an improvement guide for the MSFsc type next.

According to an embodiment of the present invention, the analysis unit 140 may receive feedback on the sequentially provided improvement guides from the user through the feedback receiving unit 150. For example, when the received feedback includes a positive response to the sequentially provided improvement guides, the analysis unit 140 may simultaneously provide the improvement guides for all types.

According to an embodiment of the present invention, when the received feedback includes a negative response to the sequentially provided improvement guides, the analysis unit 140 may adjust the range for each type of the improvement guides and sequentially provide the adjusted improvement guides for each type.

According to an embodiment of the present invention, when the sleep type changes from ‘raccoon type’ to ‘fox type’, the analysis unit 140 may determine to change the SRI type one step and change the MSFsc type two steps. At this point, the analysis unit 140 may provide the improvement guide for the SRI type of which the change in the step is small, before the improvement guide for the MSFsc type. Accordingly, the user may be provided with the improvement guides, starting from an improvement guide with less change and then an improvement guide with greater change.

A system for providing sleep state analysis information and an improvement guide according to the present invention and a method therefor have been described above. Meanwhile, the present invention is not limited to the specific embodiments and application examples described above, and it goes without saying that various modified embodiments can be made by those skilled in the art without departing from the gist of the present invention claimed in the claims, and the modified embodiments should not be understood as being distinguished from the technical spirit or prospect of the present invention.

In particular, the components that implement the technical features of the present invention included in the block diagrams and flowcharts illustrated in the drawings attached to this specification mean logical boundaries between the components. However, according to an embodiment of software or hardware, the illustrated components and their functions are implemented in the form of standalone software modules, monolithic software structures, codes, services, and combinations thereof, and as they are stored in a medium that can be executed in a computer having a processor capable of executing the stored program codes, instructions, and the like to implement the functions, all of these embodiments should also be construed as falling within the scope of the present invention.

Accordingly, although the attached drawings and description thereof illustrate technical features of the present invention, they should not be simply inferred unless specific arrangements of software for implementing such technical features are explicitly mentioned. That is, various embodiments as described above may exist, and since such embodiments can be modified partly while having the same technical features as the present invention, they should also be construed as falling within the scope of the present invention.

In addition, although the flowcharts illustrate operations in the drawings in a particular order, this is merely intended to achieve the most desirable results and should not be construed as requiring execution of the operations in the illustrated particular or sequential order or requiring execution of all the illustrated operations. In specific cases, multitasking and parallel processing may be advantageous. Furthermore, separation of various system components of the embodiments described above should not be construed as requiring such separation in all embodiments, and it should be understood that it is general for the program components and systems described above to be integrated together in a single software product or packaged in a plurality of software products.

### DESCRIPTION OF SYMBOLS

100: System for providing sleep state analysis
information and an improvement guide
110: Layer unit
120: Detection unit
130: Memory unit
140: Analysis unit
150: Feedback receiving unit

1. A system for providing sleep state analysis information and an improvement guide, the system comprising:
at least one layer unit having a plurality of components;
a detection unit provided in the layer unit to detect biometric information of a user;
a memory unit for storing the biometric information of the user detected by the detection unit; and
an analysis unit for analyzing a sleep state of the user on the basis of the biometric information of the user stored in the memory unit, and generating sleep state analysis information of the user.

2. The system according to claim 1, wherein the analysis unit analyzes the biometric information of the user stored in the memory unit in a short term, calculates a result thereof as a numerical index, and generates the sleep state analysis information of the user by including the numerical index.

3. The system according to claim 1, wherein the analysis unit analyzes the biometric information of the user stored in the memory unit in a long term, determines a sleep state type of the user on the basis of a difference in the sleep state during an analysis period, and generates the sleep state analysis information of the user by including the sleep state type.

4. The system according to claim 1, wherein the sleep state analysis information of the user analyzed by the analysis unit is stored in the memory unit.

5. The system according to claim 4, wherein, the sleep state analysis information of the user analyzed by the analysis unit is stored in the memory unit to be mapped to each of a plurality of users.

6. The system according to claim 5, wherein the analysis unit generates an improvement guide that can change the sleep state of the user on the basis of the biometric information of the user and the sleep state analysis information of the user stored in the memory unit.

7. A method of providing sleep state analysis information and an improvement guide, which is performed in a system including a layer unit, a detection unit, a memory unit, and an analysis unit to provide the sleep state analysis information and the improvement guide, the method comprising the steps of:
detecting biometric information of a user from the detection unit provided in the layer unit;
storing the biometric information of the user detected by the detection unit in the memory unit; and
analyzing a sleep state of the user on the basis of the biometric information of the user stored in the memory unit, and generating sleep state analysis information of the user, by the analysis unit.
